(19) United States
(12) Patent Application Publication
(10) Pub. No.: US 2023/0317215 A1
(43) Pub. Date: Oct. 5, 2023

(54) MACHINE LEARNING DRIVEN AUTOMATED DESIGN OF CLINICAL STUDIES AND ASSESSMENT OF PHARMACEUTICALS AND MEDICAL DEVICES

(71) Applicant: Pienomial Inc., Rockville, MD (US)

(72) Inventors: Ranjana GHOSH, Rockville, MD (US); Omkar Krishnat PATIL, Kolhapur (IN)

(73) Assignee: Pienomial Inc., Rockville, MD (US)

(21) Appl. No.: 17/709,349

(22) Filed: Mar. 30, 2022

(51) Int. Cl. G16H 10/20; G16H 70/40; G16H 15/00

(52) U.S. Cl. CPC G16H 10/20; G16H 70/40; G16H 15/00

(57) ABSTRACT

A data processing system implements receiving a set of parameters associated with a first clinical trial, the parameters identifying one or more pharmaceuticals and/or medical conditions; identifying first documents associated with one or more second clinical trials from databases of clinical trials, new drug applications, drug label information, or a combination thereof, based on the parameters; obtaining electronic copies of the first documents; analyzing the electronic copies using a first set of models configured to identify relevant portions of the electronic copies based on a document type associated with each of the electronic copies; analyzing the relevant portions of the electronic copies using a natural language processing model to extract information; collating the information extracted from the relevant portions of the electronic copies to produce prediction information related to the first clinical trial; and analyzing the prediction information to generate one or more reports providing information for assessing aspects of the first clinical trial.

### BACKGROUND

Pharmaceutical, biotech, and medical device companies invest significant amounts of time and resources in designing clinical studies and/or conducting assessments of the risk of such studies for pharmaceuticals and medical devices. These companies analyze vast amounts of data in order to understand new business development scenarios wherein they may utilize their products, to understand their competitors and products being developed by them, operational changes and risks associated with the domain in which they are operating, and the changing landscape of expert pools who have developed and are continuing to develop important domain knowledge.

The pharmaceutical, biotech, and medical device companies invest significant amounts of time and resources to perform these studies and assessments. A typical project may span many months and involve hundreds of work hours by personnel within these companies and/or by outside consultants to acquire, assess, compare, and analyze many thousands of documents. Numerous data sources are involved, including but not limited to press releases and articles regarding competitors and competing products, documents submitted to government regulatory agencies both domestically and internationally, journal articles, and published patent applications and issued patents from across the world. Acquiring, assessing, comparing, and analyzing these large volumes of data is an expensive, labor intensive, and error prone process. The team undertaking the project may easily overlook important information sources, inadvertently omit important analysis, and/or simply make errors while undertaking such an intensive project. Such errors or omissions may result incur significant costs. For example, errors associated with the testing of a single drug, group of drugs, or medical device may cost the company many tens of thousands of U.S. dollars or the equivalent thereof. Hence, there is a need for improved systems and methods of automating the acquisition and assessment of data for designing clinical studies and/or for conducting assessments of the risks involved with such studies.

### SUMMARY

An example data processing system according to the disclosure may include a processor and a machine-readable medium storing executable instructions. The instructions when executed cause the processor to perform operations including receiving a set of parameters associated with a first clinical trial, the parameters identifying one or more pharmaceuticals, one or more medical conditions, or both; identifying first documents associated with one or more second clinical trials based on the parameters associated with the first clinical trial from databases of clinical trials, new drug applications, drug label information, or a combination thereof; obtaining electronic copies of the first documents; analyzing the electronic copies using a first set of models configured to identify relevant portions of the electronic copies based on a document type associated with each of the electronic copies; analyzing the relevant portions of the electronic copies using a natural language processing model to extract information from the relevant portions of the electronic copies; collating the information extracted from the relevant portions of the electronic copies to produce prediction information related to the first clinical trial; and analyzing the prediction information to generate one or more reports providing information for assessing aspects of the first clinical trial.

An example method implemented in a data processing system for providing clinical trial recommendations includes receiving a set of parameters associated with a first clinical trial, the parameters identifying one or more pharmaceuticals, one or more medical conditions, or both; identifying first documents associated with one or more second clinical trials based on the parameters associated with the first clinical trial from databases of clinical trials, new drug applications, drug label information, or a combination thereof; obtaining electronic copies of the first documents; analyzing the electronic copies using a first set of models configured to identify relevant portions of the electronic copies based on a document type associated with each of the electronic copies; analyzing the relevant portions of the electronic copies using a natural language processing model to extract information from the relevant portions of the electronic copies; collating the information extracted from the relevant portions of the electronic copies to produce prediction information related to the first clinical trial; and analyzing the prediction information to generate one or more reports providing information for assessing aspects of the first clinical trial.

An example machine-readable medium on which are stored instructions. The instructions when executed cause a processor of a programmable device to perform operations of receiving a set of parameters associated with a first clinical trial, the parameters identifying one or more pharmaceuticals, one or more medical conditions, or both; identifying first documents associated with one or more second clinical trials based on the parameters associated with the first clinical trial from databases of clinical trials, new drug applications, drug label information, or a combination thereof; obtaining electronic copies of the first documents; analyzing the electronic copies using a first set of models configured to identify relevant portions of the electronic copies based on a document type associated with each of the electronic copies; analyzing the relevant portions of the electronic copies using a natural language processing model to extract information from the relevant portions of the electronic copies; collating the information extracted from the relevant portions of the electronic copies to produce prediction information related to the first clinical trial; and analyzing the prediction information to generate one or more reports providing information for assessing aspects of the first clinical trial.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

The drawing figures depict one or more implementations in accord with the present teachings, by way of example only, not by way of limitation. In the figures, like reference numerals refer to the same or similar elements. Furthermore, it should be understood that the drawings are not necessarily to scale.

FIG. 1 is a diagram showing an example computing environment in which the techniques disclosed herein may be implemented.

FIG. 2 is a diagram of an example implementation of the clinical trial design and assessment service.

FIG. 3 is a flow chart of an example process for automatically identifying and analyzing data that may be used to provide recommendations for generating a clinical study and/or for conducting the assessments of the risks involved with such a study.

FIG. 4 is a diagram showing an example of a document for which a machine-learning model or a rule-based model may be developed according to the techniques provided.

FIG. 5 is a diagram showing an example of another document for which a machine-learning model or a rule-based model may be developed according to the techniques provided.

FIG. 6 is a diagram of an example user interface for performing a query that may be implemented by the clinical trial design and assessment service.

FIGS. 7A, 7B, 7C, 7D, and 7E are diagrams of an example user interface that provides visualizations of the data generated by the visualization unit of the clinical trial design and assessment service.

FIG. 8 is a diagram of an example timeline that may be generated by the visualization unit of the clinical trial design and assessment service.

FIG. 9 is a diagram showing a comparison of the timelines for multiple drugs.

FIG. 10 is a flow chart of an example process for providing clinical trial recommendations.

FIG. 11 is a block diagram showing an example software architecture, various portions of which may be used in conjunction with various hardware architectures herein described, which may implement any of the described features.

FIG. 12 is a block diagram showing components of an example machine configured to read instructions from a machine-readable medium and perform any of the features described herein.

### DETAILED DESCRIPTION

In the following detailed description, numerous specific details are set forth by way of examples in order to provide a thorough understanding of the relevant teachings. However, it should be apparent that the present teachings may be practiced without such details. In other instances, well known methods, procedures, components, and/or circuitry have been described at a relatively high-level, without detail, in order to avoid unnecessarily obscuring aspects of the present teachings.

Techniques for automating the acquisition and assessment of data for designing clinical studies, comparing outcomes of other historic studies and their market performance and/or for conducting assessments of the technical and business risks involved with such studies are described. These techniques provide a technical solution to the problem of accurately acquiring, assessing, comparing, and analyzing the large volumes of data associated with such projects in a timely manner. The techniques herein utilized may be used to develop machine-learning and/or rules-based models that may rapidly identify and analyze large volumes of data to automatically generate context-based recommendations for designing a clinical study and/or for conducting assessments of the risks associated with a study. These techniques may provide significant cost saving, time savings, and labor savings compared with the current manual and labor-intensive techniques. The techniques provided herein may be used to acquire and analyze data in minutes that would have previously taken a team of analysts hundreds of hours to complete using the current manual and labor-intensive techniques. These and other technical benefits of the techniques disclosed herein will be evident from the discussion of the example implementations that follow.

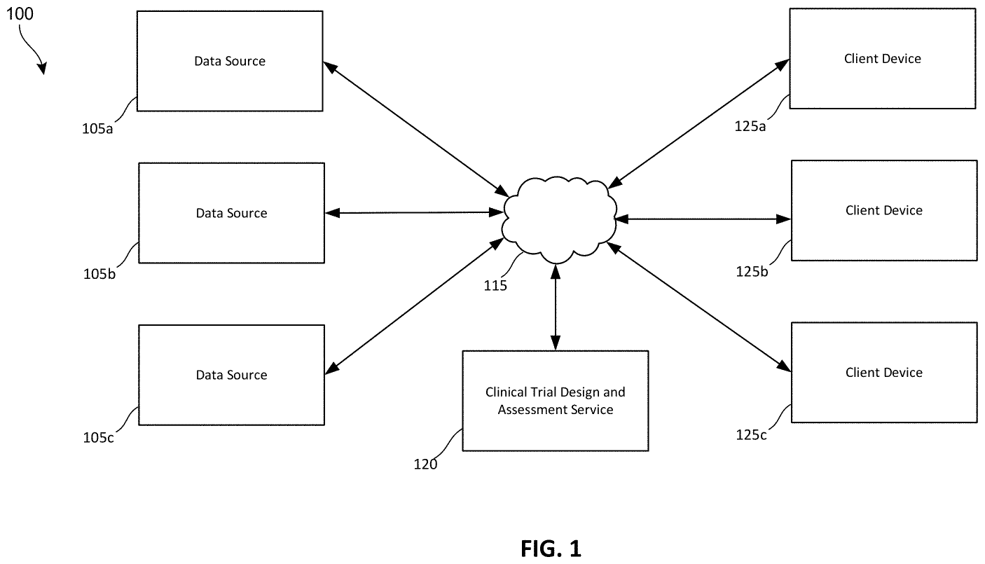

FIG. 1 is a diagram showing an example computing environment 100 in which the improved techniques for automating the acquisition and assessment of data for designing clinical studies and/or for conducting assessments of the risks involved with such studies may be implemented. The computing environment 100 may include a clinical trial design and assessment service 120 that implements techniques described herein. The example computing environment 100 may also include one or more client devices, such as the client devices 125a, 125b, and 125c. The client devices 125a, 125b, and 125c may communicate with the clinical trial design and assessment service 120 and/or the data sources 105a, 105b, and 105c (referred to collectively as data sources 105) via the network 115. The data sources 105a, 105b, and 105c may also communication with the clinical trial design and assessment service 120 via the network 115. The network 115 may be a dedicated private network and/or the combination of public and private networks commonly referred to as the Internet.

In the example shown in FIG. 1, the clinical trial design and assessment service 120 is implemented as a cloud-based service or set of services. The clinical trial design and assessment service (CTDAS) 120 is configured to facilitate the optimization of clinical studies for pharmaceuticals and/or medical devices. The CTDAS 120 is configured to receive user query parameters and to automatically identify and analyze relevant documents based on these query parameters. As will be discussed in greater detail in the examples which follow, the documents may be structured or unstructured documents. Structured documents, as used herein, refer to a document that includes some method of markup to identify elements of the document as having a specified meaning. The structured documents may be available in various domain-specific schemas, such as but not limited to Journal Article Tag Suite (JATS) for describing scientific literature published online, Text Encoding Initiative (TEI), and Extensible Markup Language (XML). Unstructured documents, also referred to as “free-form” documents herein, are documents that do not include such markup to identify the components of the documents. The CTDAS 120 may be configured to analyze both structured and unstructured documents obtained from the various data sources, such as the data sources 105a, 105b, and 105c. The CTDAS 120 may include one or more natural language processing (NLP) models configured to analyze the documents obtained from the various data sources and to extract information from these documents. The CTDAS 120 may also collate the information obtained from the documents, assess contextual relationships and patterns in the documents, and recommend context-based actions based on theses contextual relationships and patterns. Additional details of these features of the CTDAS 120 are provided in the examples which follow.

The data sources 105a, 105b, and 105c may be services that provide access to electronic versions of various types of data content that may be analyzed by the CTDAS 120 to provide guidance for optimizing clinical studies. The data sources may provide electronic copies of various types of content, including but not limited to press releases, news articles, documents submitted to regulatory agencies both domestically and internationally, journal articles, and published patent applications and issued patents both domestic and international. The data sources 105a, 105b, and 105c may include free data sources, subscription data sources, or a combination thereof. Whereas the example implementation shown in FIG. 1 includes three data sources, other implementations may include a different number of data sources. Furthermore, the data sources from which documents are acquired by the CTDAS 120 for a particular clinical study may depend, at least in part, on the parameters of the clinical study. For example, the CTDAS 120 may obtain documents from a first set of journals for a clinical study associated with a new drug and from a second set of journals for a clinical study associated with a new medical device.

The client devices 125a, 125b, and 125c (referred to collectively as client device 125) are computing devices that may be implemented as a portable electronic device, such as a mobile phone, a tablet computer, a laptop computer, a portable digital assistant device, and/or other such devices. The client device 125 may also be implemented in computing devices having other form factors, such as a desktop computer and/or other types of computing devices. While the example implementation illustrated in FIG. 1 includes three client devices, other implementations may include a different number of client devices that may utilize the services provided by the CTDAS 120. Furthermore, in some implementations, some features of the services provided by the CTDAS 120 may be implemented by a native application installed on the client device 125, and the native application may communicate with the data sources 105a, 105b, and 105c and/or the CTDAS 120 over a network connection to exchange data with the data sources 105a, 105b, and 105c, and/or to access features implemented on the data sources 105a, 105b, and 105c and/or the CTDAS 120. The native application may generate various types of telemetry information that may be sent to the CTDAS 120 for collection and processing. In some implementations, the client device 125 may include a native application that is configured to communicate with the CTDAS 120 to provide visualization and/or reporting functionality.

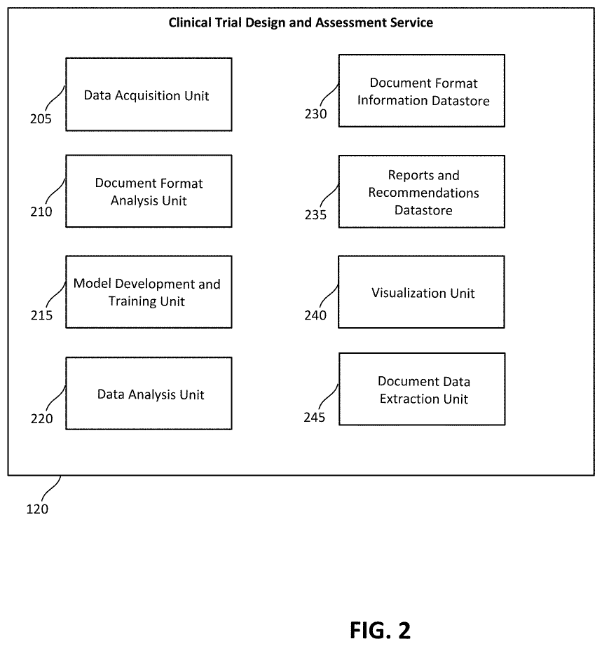

FIG. 2 is a diagram of an example implementation the CTDAS 120. The CTDAS 120 may include a data acquisition unit 205, a document format analysis unit 210, a model development and training unit 215, a data analysis unit 220, a document information datastore 230, a reports and recommendations datastore 235, a visualization unit 240, and a document data extraction unit 245.

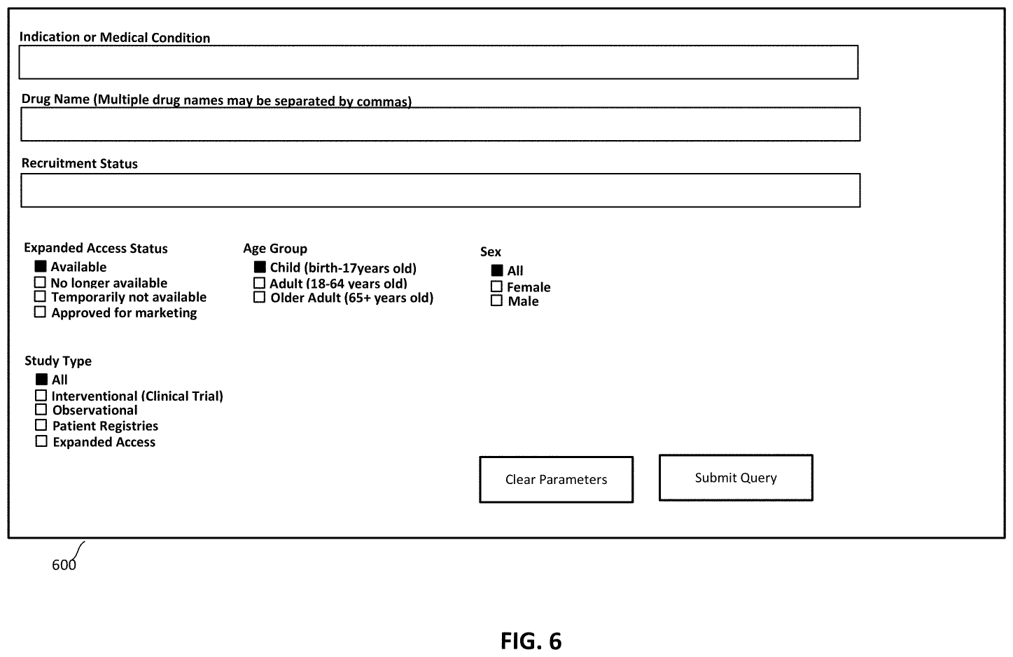

The data acquisition unit 205 may be configured to receive parameters for a clinical study to be analyzed by the CTDAS 120 and obtain documents from the data sources 105a, 105b, and 105c to be analyzed by other components of the CTDAS 120. FIG. 6 shows an example user interface 600 that may be provided by the visualization CTDAS 120 for conducting research regarding a clinical study for a drug or drugs for one or more specified medical conditions or indications. An indication, as used herein, refers to a symptom that suggests the need for a certain medical treatment. The user may enter one or more specified medical conditions or indications and/or one or more drugs for which the CTDAS 120 will search for relevant documents to be analyzed. For example, a user interested in creating a clinical study related to multiple sclerosis may enter “multiple sclerosis” in the indication or medical condition field. The user may also enter one or more drugs of interest into the drug name field to limit the search and analysis to those specific drugs. Other parameters may also be input, such as the recruitment status of clinical trials for this medical condition and/or the drugs specified, the age group of the study participants, the sex of the study participants, and/or other such parameters. The user may also enter the name of one or more drugs without entering a medical condition to obtain an analysis of various clinical trails using the the specified one or more drugs without limiting the analysis to specific medical conditions or indications. The user interface 600 may include additional parameters instead of or in addition to the example parameters shown in FIG. 6. Furthermore, the CTDAS 120 may include similar interfaces for other types of studies. For example, the CTDAS 120 may also provide a user interface for medical device studies that allows a user to enter parameters appropriate for that type of study. The user may click on or otherwise activate the “submit query” button on the user interface 600 to cause the data acquisition unit 205 to obtain documents from one or more data sources, such as the data sources 105a, 105b, and 105c.

The document format analysis unit 210 may be configured to analyze the various types of documents that may be obtained from the data sources 105a, 105b, and 105c using one or more machine learning and/or rules-based models configured to identify relevant sections of these documents that contains information that may be extracted from the documents by the document data extraction unit 245. Many of the documents obtained from the data sources 105a, 105b, and 105c may be unstructured documents that do not have any markup that identifies the location of information within the document. Furthermore, these documents may be lengthy and include a considerable amount of information that may not be directly relevant to the analysis to be performed. For example, the documents for a clinical study for a single drug are often between 50 to 75 pages in length. Much of the information included in the document may not be relevant to the clinical study analysis, and the information that is relevant may be scattered throughout the document. Analyzing the entire document with a natural language processing model is impractical and would consume an extensive amount of time and computing resources to process the large number of documents that may be analyzed for a particular study. The document format analysis unit 210 provides a technical solution to this technical problem by building machine learning models and/or rules-based models that may be used to first identify the relevant portions of the unstructured documents. A technical benefit of this approach is that the document format analysis unit 210 facilitates the standardization of the processing of the various types of documents that may be obtained from the data sources 105a, 105b, and 105c to efficiently identify and extract data from relevant portions of the unstructured documents, which may significantly reduce the computing time and resources required to analyze the documents.

The document format analysis unit 210 may be configured to use the one or more machine learning models and/or rules-based models when analyzing documents to identify relevant sections of structured or unstructured documents. The document format analysis unit 210 may be configured to use various types of deep learning models to extract the format information from structured or unstructured documents, such as but not limited to natural language processing algorithms or models, Generative Pre-trained Transformer 3 (GPT-3), and/or various pattern recognition algorithms. The document information datastore 230 may include information mapping a particular machine learning or rules-based model that may be used to analyze a particular type of document. The models may be created using the model development and training unit 215 to create new model and/or to update existing models to handle new types of documents to be analyzed. Some models used to analyze the documents may be pretrained. The document format analysis unit 210 may be configured to identify the type of document using metadata associated with the document, by analyzing the contents of the document, by analyzing a file type extension of a filename of the document, and/or by providing the document as an input to a machine learning model configured to receive a document as an input and to output of a prediction of the type of the document. The document format analysis unit 210 may utilize rules-based models on structured documents to identify markup elements. These rules-based models may identify the location of specific tags that may be used to identify content items within the structured document to identify the relevant sections of the structured document.

The document format analysis unit 210 may output information identifying relevant sections of a document to the document information datastore 230. The document format analysis unit 210 may associate a unique identifier associated with a document with the one or more relevant section identifiers that identify the relevant sections of the document. The relevant section identifiers may vary depending upon the implementation and the type of document. For example, the relevant section identifiers for a document formatted into paragraphs may be paragraph numbers. In some documents, the relevant section identifiers may be section headers for documents that include such headers to subdivide the document into sections. In other documents, the relevant section identifiers may be identified by identifying a range of characters that comprise the relevant section of the document. Other such types of section identifiers may also be used by the document format analysis unit 210 to denote which portion or portions of the document may include relevant information.

The model development and training unit 215 is configured to develop the models used by the document format analysis unit 215 to analyze the structured and unstructured documents. Many documents of the documents that may be analyzed by the CTDAS 120 are unstructured documents that lack the markup information of structured documents that provide meaning to elements of the document. However, the model development and training unit 215 may be used to train one or more machine learning models and/or to develop one or more rules-based models that are configured to identify the locations of information of interest within the unstructured documents.

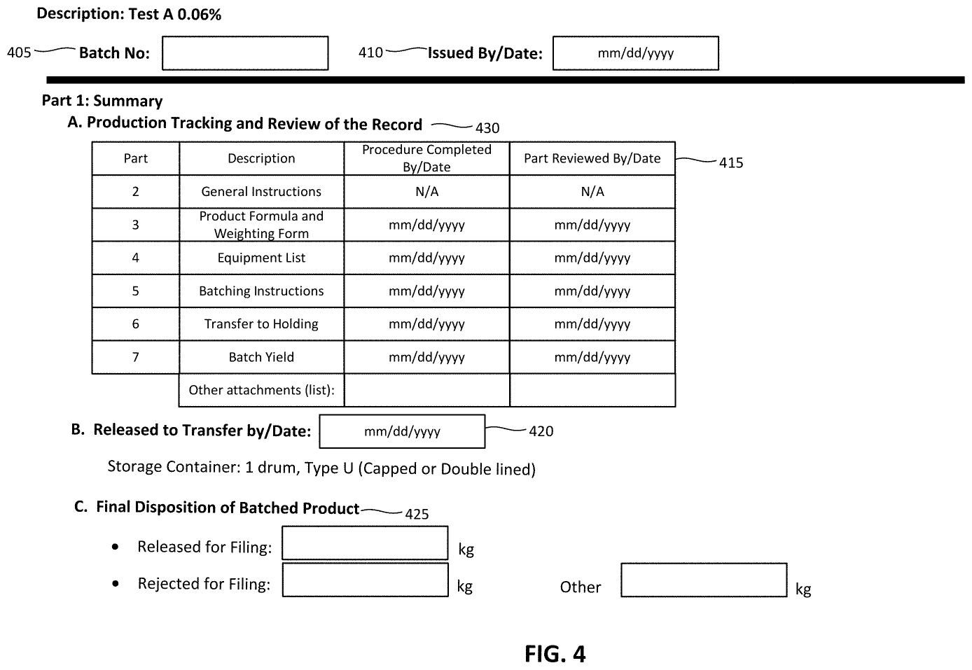

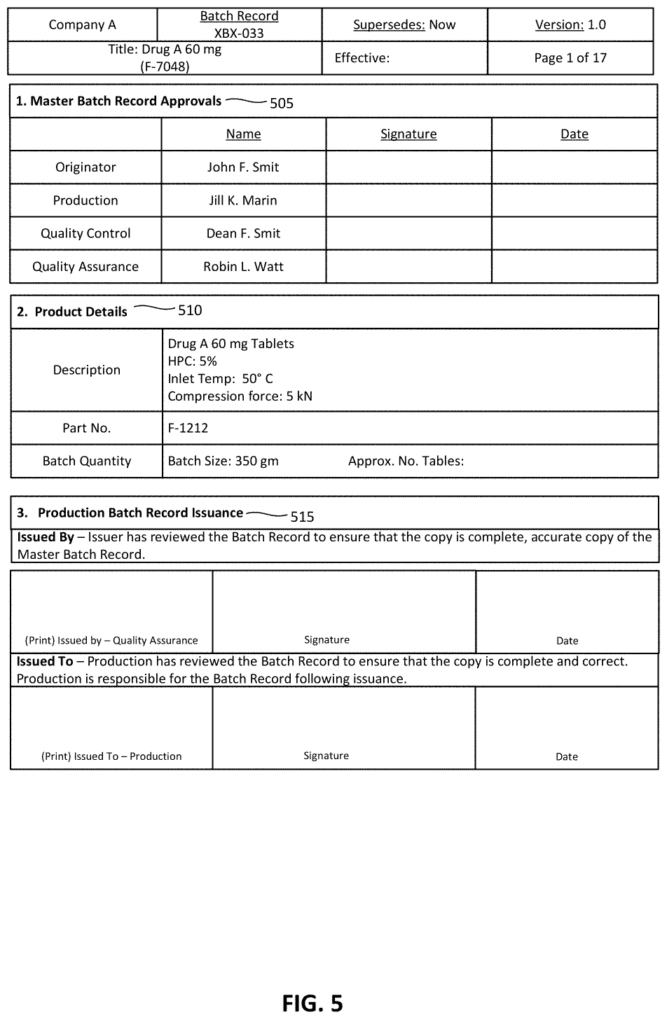

The document format information datastore 230 may include information that identifies the key terms, parameters, and/or variables that are included in a particular type of document to be analyzed by the CTDAS 120. These key terms may be identified by a user and entered via a user interface provided the CTDAS 120. The model development and training unit 215 may use this information for a respective document type to create a model that can determine a location of these key terms, parameters, and/or variables with a document of that document type. The location of these key terms, parameters, and/or variables may be determined based on “landmarks” in the document. Examples of landmarks include field labels, section headers, and/or other textual content that is typically located proximate to the content of interest to be extracted from the document. The locations of such landmarks can be determined relative to one another and a key term, parameter, and/or variable of interest to be extracted from a document. FIGS. 4 and 5, described in detail below, provide examples of unstructured documents that include such landmarks.

The model development and training unit 215 may be configured to utilize a pattern identification algorithm to identify the patterns of such landmarks relative to a key term, parameter, and/or variable of interest. The model development and training unit 215 may be configured to utilize various types of pattern recognition algorithms. One such pattern recognition algorithm uses Delaunay Triangulation Analogy (DTA) to generate relational pattern information for the document. DTA may be used to match concepts across documents and hence identify location of relevant data. This geometric matching may be applied to the location of key terms, parameters, and/or variables within the document based on the relative to landmarks in the textual content of the unstructured document. Another pattern recognition algorithm may utilize Voronoi diagrams analogy. Other types of pattern recognition algorithms may also be used. The model development and training unit 215 may use such a pattern recognition approach to generate training data for a machine-learning model and/or for generating the rules for a rules-based model that can analyze a specific type of document and output information identifying the relevant sections of the documents to be analyzed by the CTDAS 120.

The document data extraction unit 245 may be configured to analyze the relevant sections of the documents identified by the document format analysis unit 210 to extract information from the documents. As discussed above, the document format analysis unit 210 may store the information identifying the one or more relevant sections of the document. The document data extraction unit 245 may access the relevant section information for a document being analyzed and analyze those sections of the document with one or more natural language processing (NLP) models to extract textual content from the document that may be analyzed by the data analysis unit 220. The document data extraction unit 245 may be configured to use various deep learning models to analyze the textual content, such as but not limited to GPT-3 and GPT-J. Other NLP and/or deep learning models may be used in other implementations. A technical benefit of this approach is that the NLP models may be trained on data having a standardized format. The document data extraction unit 245 may be configured to extract the data from the relevant sections of the documents being analyzed and to convert the data to the standardized format. The inferences output by the NLP models may be significantly improved because the data input to the models is in the same standardize format used for training the models.

The information extracted by the one or more NLP models may be stored in the document information datastore 230 by the document data extraction unit 245. The NLP models used to extract textual content from the documents may be very computationally intensive. A technical benefit of applying the NLP model or models only to the portions of the document that have been identified as being relevant is that the amount of time and computational resources required to extract the relevant information from the document may be significantly decreased. As a result, the CTDAS 120 may rapidly analyze the documents associated with a clinical study for a drug or drugs for one or more specified medical conditions or indications. Consequently, the CTDAS 120 may reduce the amount of time to perform such an analysis from the hundreds of hours required using current methods to a matter of minutes.

The data analysis unit 220 may be configured to analyze and collate the data extracted from the documents by the document data extraction unit 245. The data analysis unit may be configured to collate data based on the medical conditions and/or indications associated with the data and/or the drug or medical device used to treat the medical conditions and/or indications. The data analysis unit 220 may be configured to cluster documents and data sets based on trends of parameters acquired from these documents using an Elasticsearch model. These parameters may include but are not limited to phase of trial, trends in investment, trends in stock price, trends in business and organization relationships, trends in patents filed, trends in the structure of clinical studies, and trends in the results of clinical studies. Elasticsearch provides tools for metrics aggregation, buckets aggregations for analyzing distinct categories in the data or for comparing these categories, and pipeline aggregations in which output produced by other aggregations have statistics and/or granular metrics added.

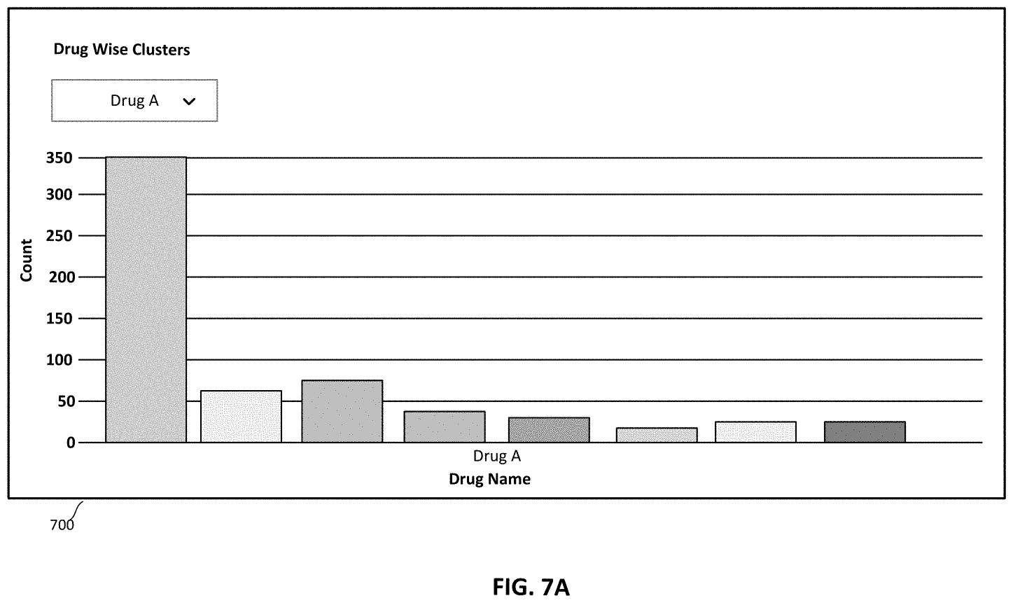

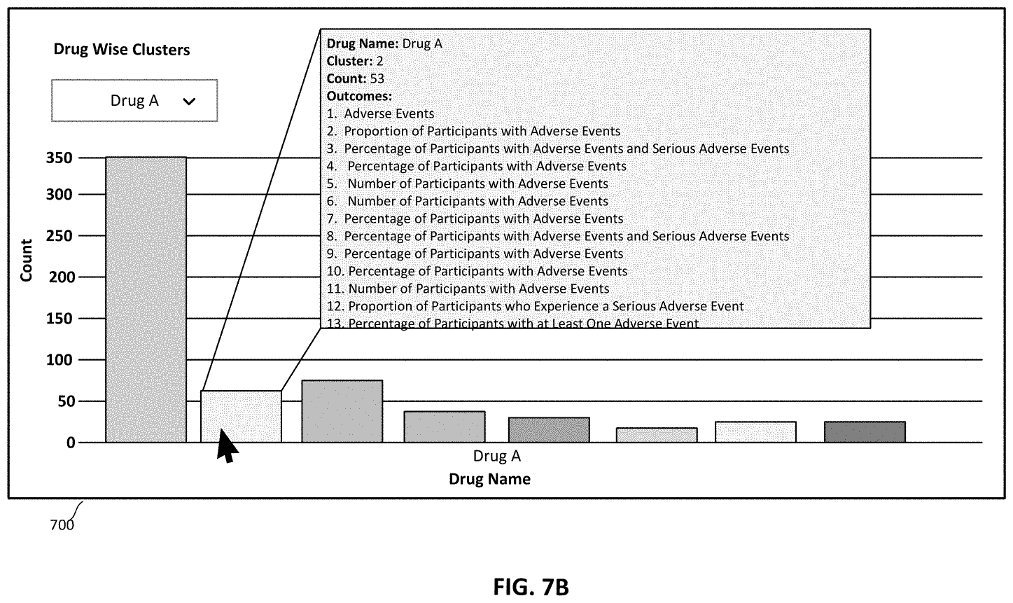

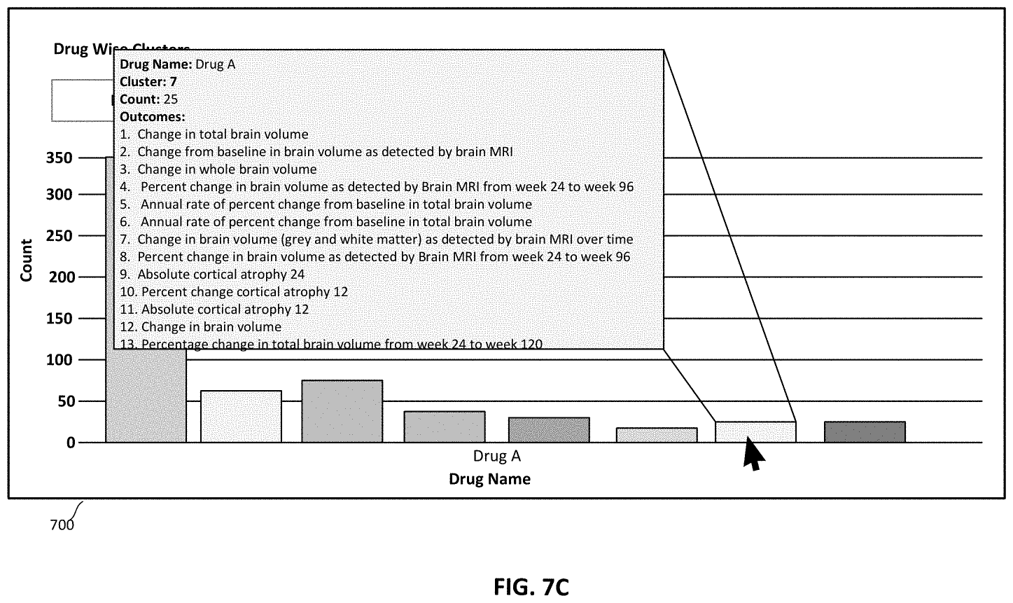

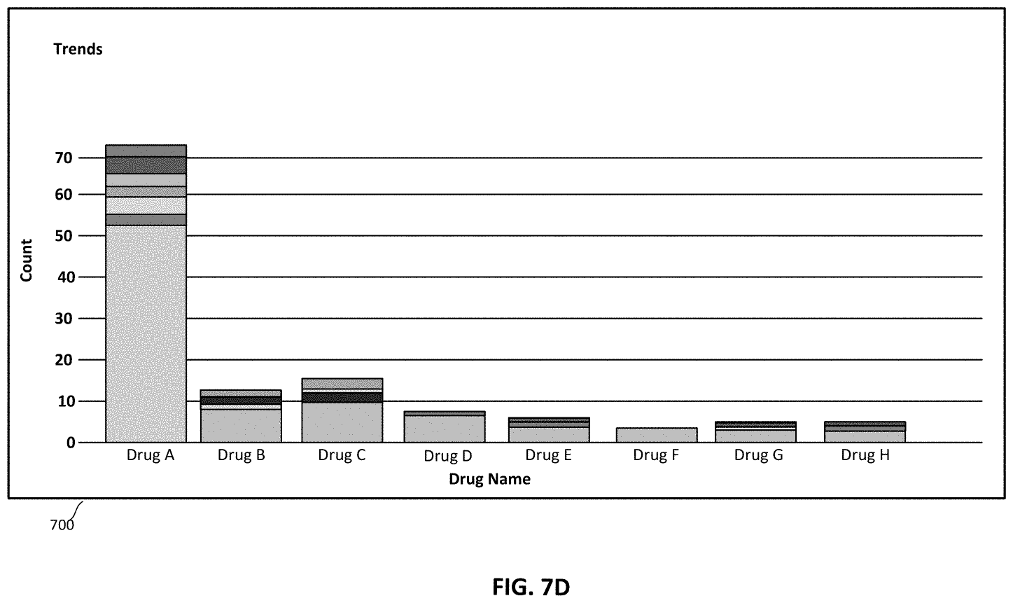

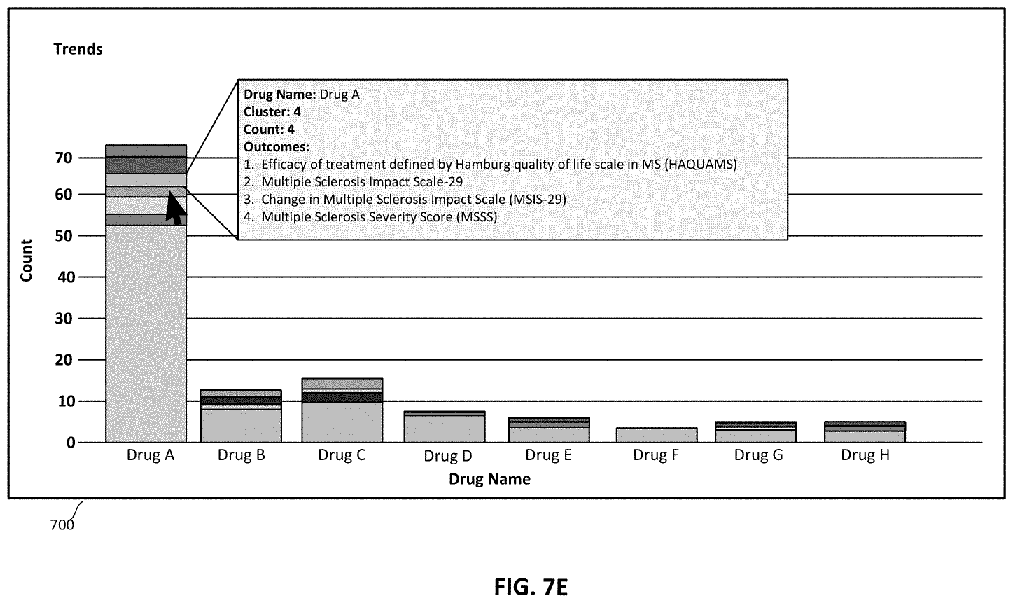

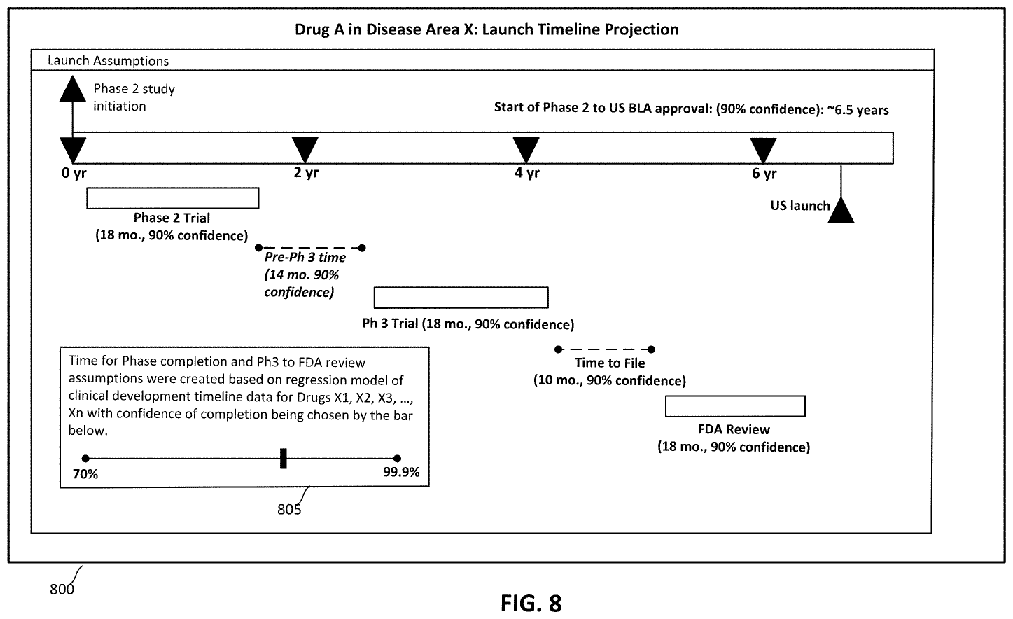

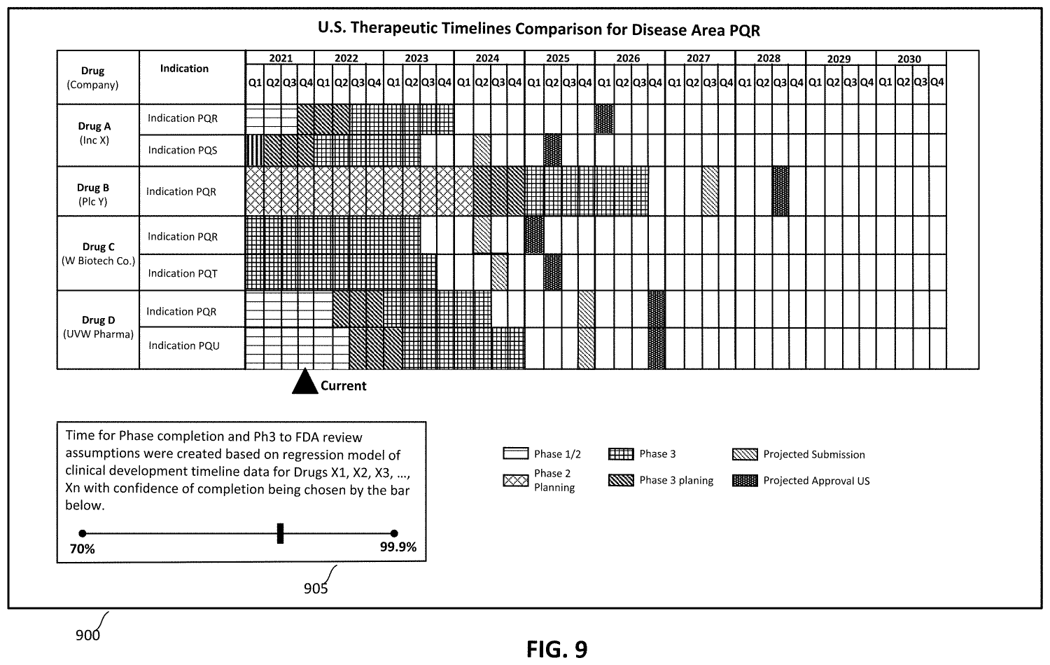

The data analysis unit 220 may be configured to automatically generate various types of context-based recommendations for designing a clinical study and/or for conducting assessments of the risks associated with such a study. These reports may be referred to by various groups within a pharmaceutical or medical device manufacturer to determine whether to conduct clinical studies for the pharmaceutical or medical device. Some examples of the types of reports that may be generated are shown at in FIGS. 7A-7E, 8, and 9. FIGS. 7A-7C show an automated categorization of clinical study endpoints across all clinical studies for a specific drug for a specific disease. FIGS. 7D and 7E show an automated categorization of clinical study endpoints across all drugs of interest for a specific disease. FIG. 8 is a diagram of an example estimated clinical development timeline 800 that may be generated by the visualization unit of the clinical trial design and assessment service. FIG. 9 is a diagram of a user interface 900 showing a comparison of the timelines for multiple drugs. Additional details of FIGS. 7A-7E, 8, and 9 are provided below.

Other types of presentations or reports may also be generated instead of or in addition to one or more of these example reports. Presentations or reports may be designed for a specific audience looking for specific insights. The reports and recommendations datastore 235 may store predetermined templates which may include figures and/or graphs which may be automatically generated by the visualization unit 240 using the various techniques described herein. The presentations or reports may also include qualitative and quantitative indications. The quantitative text may be generated by the data analysis unit 220 based on qualitative analysis (e.g., “6 new drugs started clinical trials in 2021”). The qualitative text may include terms such as but not limited to increase, decrease, inflections, and instability. The text may be generated by rule-based algorithms (e.g., “there was a 30% increase in the number of approvals in 2022”).

The data analysis unit 220 may generate an estimated timeline for a clinical trial based on timeline information associated with one or more second clinical trials. The data analysis unit 220 may utilize machine learning models trained on timeline information from previously conducted clinical trials to provide predictions for timing and length of the various phases of a subsequent clinical trial. The data analysis unit 220 may also generate an assessment of the endpoints in one or more second clinical trials relevant to a specified clinical trial. The results of these studies from earlier phases of the drugs being tested, evolution of endpoints in these trials, and a comparison of the endpoint outcomes based on mechanisms. The data analysis unit 220 may generate an assessment of comparative performance of drugs based on warnings, contraindications, adverse reactions, administration and safety concerns by comparing the data collected from the data sources 105a, 105b, and 105c. The data analysis unit 220 may generate an assessment of the probability of business success based on resources, patents, expertise of the organization and/or individuals in the organization, partnerships with other organizations and/or individuals, financial status of organization, and comparison with similar drug development by that organization or other organizations. The data analysis unit 220 may generate an assessment of the probability of product performance based on results from past clinical studies of given drug.

The visualization unit 240 is configured to generate graphical representation associated with the recommendations generated by the data analysis unit 220. The visualization unit 240 may be configured to generate graphs, charts, plots, and other graphical representations of the data that may assist the user in identifying various trends associated with clinical studies. Examples of such visualizations are provided in FIGS. 7A-7E, 8, and 9, which are described in detail in the examples which follow.

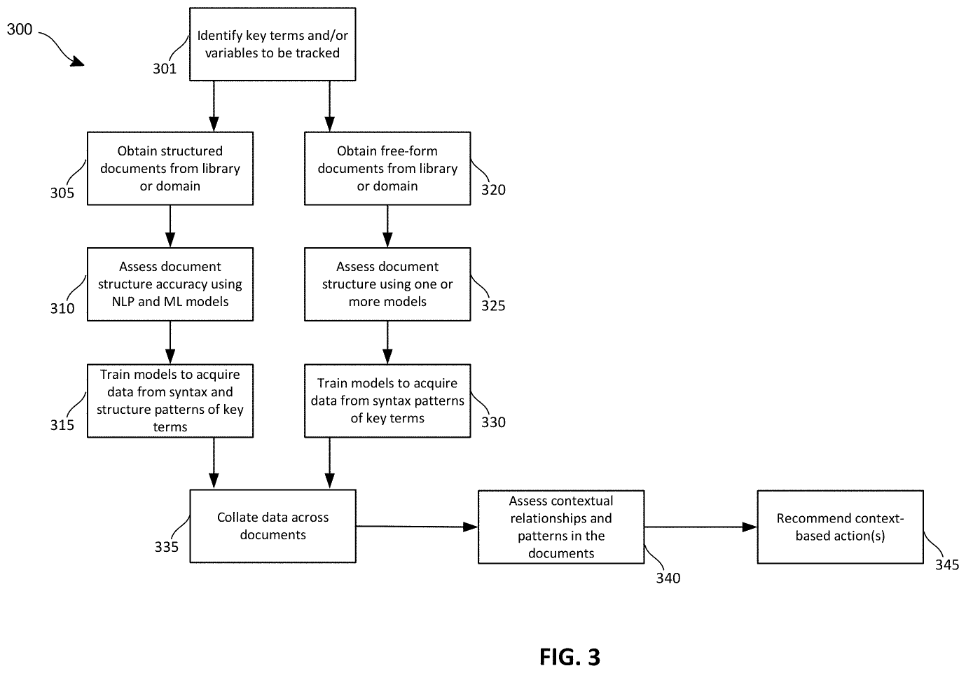

FIG. 3 is a flow chart of an example process for automatically identifying and analyzing data that may be used to provide recommendations for generating a clinical study and/or for conducting the assessments of the risks involved with such a study. FIG. 3 is an example of a process that may be implemented by the CTDAS 120.

The process 300 may include an operation 301 of identifying key terms and/or variables to be tracked. As discussed in the preceding examples, the CTDAS 120 may provide a user interface similar to the user interface 600 shown in FIG. 6 that permits a user to define the parameters, such as but not limited to one or more drug names, one or more medical conditions or indications, demographic information for study participants, and/or other such parameters. The data acquisition unit 205 of the CTDAS 120 may use these parameters to acquire documents to be analyzed from the data sources 105a, 105b, and 105c.

The process 300 may include an operation 305 of obtaining structured documents from library or domain. The data acquisition unit 205 of the CTDAS 120 may acquire structured documents that include semantic information that may be used to identify the location of information within the document that is relevant for generating the context-based recommendations for designing a clinical study and/or for conducting assessments of the risks associated with such a study.

The process 300 may include an operation 310 of assessing document structure accuracy using one or more models. The data acquisition unit 205 of the CTDAS 120 may be configured to analyze the structure of the structured document with an NLP model associated with the type of structured document being processed. The model may be configured to output a prediction that the document structure is accurate or requires attention. If the document structure is accurate, the process 300 may continue to operation 315. Otherwise, the document may be flagged as including errors. The user may be provided with a notification that the document could not be processed.

The process 300 may include an operation 315 of training one or more models to acquire data from syntax and structure patterns of key terms. The model development and training unit 215 may be configured to generate one or more machine learning and/or rules-based models that are configured to identify the location of relevant information within a structured document. The model development and training unit 215 may generate a separate model for each type of structured document.

The process 300 may include an operation 320 of obtaining free-form or unstructured documents from library or domain. Unstructured documents may comprise textual content that lacks the semantic information provided in structured documents. Unstructured documents may, in some instances, be generated by extracting the textual content of from an image or scan of a physical document.

The process 300 may include an operation 325 of assessing document structure using one or more models. The operation 325 may include analyzing the contents of the document with one or more NLP models to obtain contextual information for the textual content of the document. The structure of the document may be verified by checking the markup information to determine whether the document structure appears correct based on the information extracted from the document by the one or more NLP models. In some implementations a model specific for the document type of the document being verified may be used to verify the document structure, while other implementations may use models that are able to verify the structure of multiple types of documents. In some implementations, a rule-based model may be used to verify the structure of the document. In other implementations, a machine learning model may be trained to analyze the structure of the document.

The process 300 may include an operation 330 of training models to acquire data from the syntax pattern of key terms within the documents. The model development and training unit 215 may be configured to generate one or more machine learning and/or rules-based models that are configured to identify the location of relevant information within a document. The document data extraction unit 245 may use these models to extract relevant information from the documents. Models may be developed for each type of document that may be processed by the CTDAS 120 and the models may be refined by analyzing multiple documents of the same type and refining the model based on these documents. Different instances of the same type of document may include sections that may not be included in all instances of the document. Processing multiple instances of documents to develop training data for the ML-model or rules for a rules-based model may provide a model that provides better results predicting the relevant sections of a document.

The process 300 may include an operation 335 of collating data across documents. The data analysis unit 220 of the CTDAS 120 may be configured to analyze and collate the data extracted from both the structured and unstructured documents. As will be discussed in greater detail in the examples which follow, such as those shown in FIGS. 7A-7E, the data may be collated by drug and/or by indication or medical condition.

The process 300 may include an operation 340 of assessing contextual relationships and patterns in the documents. The data analysis unit 220 may also assess contextual relationships and identify patterns in the documents. The results of the analysis may be presented to users to assess how

The process 300 may include an operation 345 of recommending context-based actions. The CTDAS 120 may provide visualizations of the data analyzed and collated by the data analysis unit 220. Examples of such visualizations of these assessments are shown, inter alia, in FIGS. 7A-7E, 8, and 9. The CTDAS 120 may also provide tools for scenario assessment and modeling, identifying trends, action plan management, and decision optimization. Other tools for providing early error assessment and root cause assessment may also be provided by the CTDAS 120.

FIG. 4 is a diagram showing an example of an unstructured document which is a form that may be used for the approval or rejection of product batches of a pharmaceutical being tested. The unstructured document is textual content and does not include semantic tags. However, the textual content of the document includes various text labels that may be used to identify the location of relevant data within the document. For example, the batch number label 405, the issued by date label 410, and the released to transfer by data label 420 may be used to identify the location of the batch number, he issued by date of the batch, and the release to transfer by data of the batch respectively, because the labels 405, 410, and 420 are located next to the respective data element that represented by that label. The location of the data included in the table 415 may be identified based on the location of the section header 430 “A. Production Tracking and Review of the Record” and the contents of individual rows may be determined based on the contents of the “Description” column of the table. The location of “Released for Filing” and “Rejected for Filing” fields may be determined based on the location of the section header 425 and/or based on the location of the labels: “Released for Filing,” “Rejected for Filing,” and “Other” shown in FIG. 4.

FIG. 5 is a diagram showing an example of another document for which a machine-learning model or a rule-based model may be developed according to the techniques provided. The example document shown in FIG. 5 is a sample of a manufacturing batch record. Like the example shown in in FIG. 4, the document shown in FIG. 5 includes section headers 505, 510, and 515 that may be used as landmarks for locating key terms, parameters, and/or variables of interest within the document. The layout of the document shown in FIG. 5 is different from that of the document shown in FIG. 4, and the model development and training unit 215 may generate a separate model for processing documents of each type. The example documents shown in FIGS. 4 and 5 provide examples of the types of unstructured documents that may be obtained and analyzed by the CTDAS 120, but CTDAS 120 is not limited to these specified document types. The CTDAS 120 may be configured to handle other types of structured and unstructured documents.

FIGS. 7A, 7B, 7C, 7D, and 7E are diagrams of an example user interface 700 that provides visualizations of the data generated by the clinical trial design and assessment service 120. The user interface 700 may be displayed in response to the user submitting a query via the user interface 600 shown in FIG. 6.

FIGS. 7A-7C show an automated categorization of clinical study endpoints across all clinical studies for a specific drug for a specific disease. The user interface may include a dropdown that allows the user to select which drug is shown. In the example shown in FIGS. 7A-7C, Drug A has been selected. The graph shown in FIGS. 7A-7C may be used to identify which endpoints are of interest to clinical studies and new endpoints of interest in these studies. An endpoint of a clinical trial is an event or outcome that may be measured objectively to determine whether the drug being tested provides a beneficial outcome regarding the disease being treated.

FIG. 7A shows the resulting graph for Drug A. FIG. 7B shows the one of the clusters having been selected. In this example, the user may select a first cluster by positioning a user interface pointer over the cluster. In response, the user interface 700 may show additional information associated with the first cluster. FIG. 7C shows the user interface 700 shows additional information associated with a second cluster. In this example, the first cluster is a logical grouping of documents associated with adverse events that have been documented in clinical trials using Drug A for the treatment of multiple sclerosis, and the second cluster is a logical grouping of documents associated with changes in brain volume documented in clinical trials using Drug A for the treatment of multiple sclerosis.

FIGS. 7D and 7E show an automated categorization of clinical study endpoints across all drugs of interest for a specific disease. The graph shown in FIGS. 7D and 7E identifies which endpoints are of interest to clinical studies and new endpoints of interest in these studies. The graph shown in FIGS. 7D and 7E can be used to quickly identify trends in drug treatments for a specific disease. In this example, Drug A had the most activity, with over 70 study-related documents found for Drug A being used to treat multiple sclerosis. Drugs B-H appear much less frequently in the documents analyzed by the CTDAS 102. The data bar associated with each drug is broken down into multiple sections that represent clusters of documents. Each cluster is logical grouping of documents that the models used by the CTDAS 120 have determined are related. For example, FIG. 7E shows an example in which the user has selected a cluster associated with Drug A to show additional details associated with that cluster. The selected cluster is related to efficacy of Drug A in treating multiple sclerosis and the impact of this treatment on the quality of life of the patient. Other clusters may relate to other topics, such as but not limited to adverse reactions to the treatment, warning and precautions associated with the treatment, clinical endpoints or outcomes associated with the treatment, and/or other factors that may need to be taken into consideration when determining whether to conduct a clinical trial with Drug A. The user may select other clusters associated with Drug A and/or the other drugs shown to obtain additional information, which may be shown in a popup window as depicted in FIG. 7E.

FIG. 8 is a diagram of an example estimated clinical development timeline 800 that may be generated by the visualization unit of the clinical trial design and assessment service. The clinical trials are typically conducted in a multi-phase approach that typically span many years. Phase 1 typically includes a small number of healthy volunteers that test the drug for safety and tolerability of the drug at different doses. Phase 2 typically includes a larger number of test subjects and determines the efficacy and optimal dose at which the drug shows biological activity with minimal side-effects. Phase 3 typically includes an even larger number of test subjects and determines the effectiveness of the drug over current treatments. In the United States, a Biologics License Application (BLA) may be submitted to the Food and Drug Administration (FDA) to review the results of the clinical trials for a determination whether the drug may be approved to treat the illness for which the drug was tested. While the example shown in FIG. 8 is for a drug that is clinically tested and submitted for approval in the United States, a similar estimated timeline may be generated for drugs tested and submitted for approval in other countries or regions. Currently, the development of such an estimated timeline is very labor intensive and manual process in which a team of analysts search for and analyze clinical development timelines for other drugs to estimate the clinical development timeline for a drug to be tested. This process is further complicated by substantial number of parameters that may vary among clinical studies. The demographics of the participants selected to participate in the trial, the size of the study group, and/or other parameters may have a significant impact on the planning and execution of each phase of the study. Consequently, these and other factors may significantly impact how long the planning and execution of each phase take to complete.

In the example shown in FIG. 8, an example of a projected launch timeline for a Drug A for use in a disease area X is shown. In some implementations, the projected launch timeline may focus on a single disease area, such as but not limited to Multiple Sclerosis. Other implementations may focus on a different single disease area. A disease area refers to a grouping of related diseases, such as but not limited to autoimmune diseases, cardiovascular diseases, endocrine diseases, gastrointestinal diseases, neurological diseases, and/or other groups of diseases. The user may specify the parameters of the clinical studies to be conducted on this drug. In some implementations, the user may input a set of parameters to be investigated via the user interface 600 shown in FIG. 6. The user may submit queries using various permutations of the clinical studies to obtain an estimated timeline based on those parameters. For example, the user may initially limit the clinical study parameters to adult female participants to obtain a first estimated clinical development timeline. The user may then submit a second query in which the clinical study parameters have been expanded to include both male and female participants to obtain a second estimated clinical development timeline. The two timelines may be compared to provide an estimate on how changing the clinical study parameter may impact the estimated clinical development timeline. In some implementations, the CTDAS 120 may provide an interface that permits the user to submit multiple sets of clinical study parameters, and the CTDAS 120 may generate a clinical development timeline for each of the sets of clinical study parameters. The visualization unit 240 may be configured to provide a user interface that provides a comparison of the multiple clinical development timelines so that the user may more readily understand the impact of changing the clinical study parameters on the estimated timeline.

The data analysis unit 220 of the CTDAS 120 may utilize one or more machine learning models configured to predict the length and/or estimated scheduling of each of the phases of the clinical trial. In the example shown in FIG. 8, the estimated of the time from Phase 3 to FDA review is based on the clinical development of n other drugs, wherein n is a positive integer. The machine learning model may be trained to receive drug information various parameters, such as but not limited to a drug name, a condition for which the drug is tested, demographic information for the participants of the clinical study and the size of the population, criteria by which participants may be included or excluded, and/or additional parameters associated with other characteristics of the clinical study and to output a predicted scheduled for the clinical study based on these parameters.

The example user interface 800 shown in FIG. 8 includes a confidence level control 805 that allows a user to adjust a confidence value associated with the predicted timeline. A higher confidence value correlates with less risk that the predicted time will be inaccurate but may result in a much longer amount of time being predicted to complete the clinical study. A lower confidence level may run the risk of underestimating or overestimating the length time to complete the study. The confidence value may be used by the data analysis unit 220 may use the confidence level value to determine a confidence interval for the predictions used to generate the projected launch timeline that are at least correct at least a threshold percentage of times represented by the confidence level value. For example, if the user selects a confidence level value of 80%, then the projected launch timeline should be correct at least 80% of the time.

In some implementations, the models used by the data analysis unit 220 may generate output a confidence score associated with the inferences made by the models. The confidence score may be a numerical value representing an estimate of how likely the inference is correct. The data analysis unit 220 may be configured to discard inferences that fall below the confidence value specified by the user. In other implementations, the confidence values may be used to exclude data being used to generate the predictions. For example, the data analysis unit 220 may calculate a confidence interval based on the confidence value specified by the user. The confidence interval may be based on the mean, standard deviation, and sample size for the data being provided as inputs to the models used by the data analysis unit 220. The data analysis unit 220 may calculate a standard error value for sample data by dividing the standard deviation of the of the sample data by the square root of the number of data points included in the sample data. The data analysis unit 220 may the multiply the standard error value by a Z-score to obtain a margin of error value. The Z-score represents a number of standard deviations by which a data point is above the mean. The margin of error value may then be used to determine the upper and lower bounds for the confidence interval when selecting data to be provided as input. The lower bound of the confidence interval may be determined by subtracting the margin of error value from the mean, and the upper bound of the confidence interval may be determined by adding the margin of error value to the mean. Data values falling outside of the confidence interval may be discarded.

FIG. 9 is a diagram of a user interface 900 showing a comparison of the timelines for multiple drugs. Each drug may include an identifier of the particular drug, an indication or indications for which a study is being conducted to test the efficacy of the drug for that indication or indications. The timeline also includes a calendar that projects current and/or expected scheduling of each of the phases of testing associated with each of the drugs. The timeline provides information that may be used to assess the progress that competitors have made testing drugs and/or medical devices. This information may be used to assess whether competitors are ahead or lagging behind on the progress made by an organization using the CTDAS 120 to assess whether to conduct a clinical study for their own drug(s) and/or medical device(s) based on the current progress demonstrated by competitors. The user interface 900 may be generated by the visualization unit 240 of the CTDAS 120 based on the information generated by the data analysis unit 220. The approval timelines can show the progress of the clinical trials for one or more indications being treated using a particular drug. The approval timeline may include an estimated clinical timeline shown in FIG. 8 so that the user may compare the estimated clinical timeline with the timelines for other drugs. The timelines shown in FIG. 9 may be used to determine which competitors have drugs and/or medical devices being released and how this timeline compares to the drug or medical device for which the user is developing a clinical development timeline.

The example user interface 900 shown in FIG. 9 includes a confidence level control 905 that allows a user to adjust a confidence value associated with the predicted timeline in a similar manner as the confidence level control 805 shown in FIG. 8. The data analysis unit 220 may use the confidence values as discussed above with respect to FIG. 8.

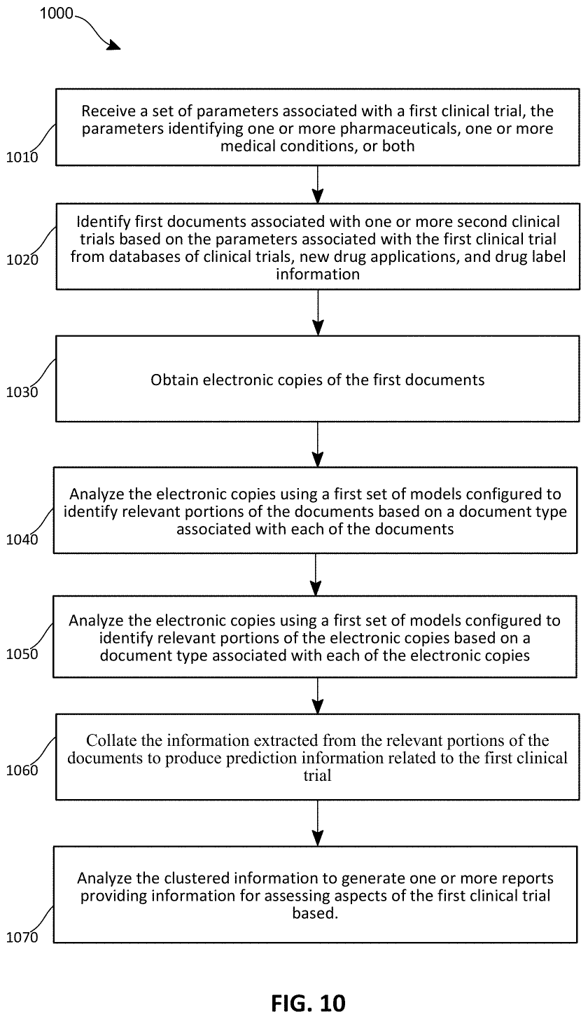

FIG. 10 is a flow chart of an example process 1000 for providing clinical trial recommendations. The process 1000 may be implemented by the clinical trial design and assessment service 120. The process 1000 may be used to implement the techniques for acquiring and analyzing clinical trial information described herein.

The process 1000 may include an operation 1010 of receiving a set of parameters associated with a first clinical trial, the parameters identifying one or more pharmaceuticals, one or more medical conditions, or both. The CTDAS 120 may provide a user interface, such as the example user interface 600 shown in FIG. 6, for receiving a set of parameters for which the user would like to obtain a clinical trial recommendation.

The process 1000 may include an operation 1020 of identifying first documents associated with one or more second clinical trials based on the parameters associated with the first clinical trial from databases of clinical trials, new drug applications, and drug label information and an operation 1030 of obtaining electronic copies of the first documents. As discussed in the preceding examples, the data acquisition unit 205 may identify and obtain electronic copies of relevant documentation from one or more of the data sources 105a, 105b, and 105c. This documentation may include information about other clinical trials that have been completed or are in progress. The CTDAS 120 may analyze this information to provide recommendations and estimates regarding the first clinical trial.

The process 1000 may include an operation 1030 of analyzing the electronic copies using a first set of models configured to identify relevant portions of the electronic copies based on a document type associated with each of the electronic copies. The document data extraction unit 245 may utilize one or more models generated by the model development and training unit 215 to identify relevant portions of documents being processed.

The process 1000 may include an operation 1040 of analyzing the electronic copies using a first set of models configured to identify relevant portions of the electronic copies based on a document type associated with each of the electronic copies. This approach may significantly reduce the amount of content from the documents that needs to be processed and analyzed by limiting the use of natural language processing models to only those portions of the textual content determined to be relevant. The models used to identify the relevant portions of the documents may utilize various pattern recognition algorithms to identify the relevant portions of the documents.

The process 1000 may include an operation 1050 of collating the information extracted from the relevant portions of the electronic copies to produce prediction information related to the first clinical trial. The data analysis unit 220 may be configured to collate the data across the documents to generate clusters of data based on the indication or medical condition being treated in a respective clinical trial, the drug or medical device used for treatment, and/or the outcome of the treatment. The data may be further clustered based on adverse reactions or conditions that occurred during the treatment and other related factors. Examples of the results of such clustering are shown at least in FIGS. 7A-7E. The data analysis unit 220 may also determine insights and predictions around probability of success, performance, timelines, costs, competitiveness and revenue of the first clinical trial.

The process 1000 may include an operation 1060 of analyzing the clustered information to generate one or more reports providing information for assessing aspects of the first clinical trial. The CTDAS 120 may be generate various types of reports that may be presented to the user via a user interface of their client device. Examples of the types of visualizations of the data that may be presented to the user are shown in FIGS. 7A-7E, 8, and 9. Other types of reports and/or visualizations may also be provided in addition to or instead of one or more of these examples.

The detailed examples of systems, devices, and techniques described in connection with FIGS. 1-10 are presented herein for illustration of the disclosure and its benefits. Such examples of use should not be construed to be limitations on the logical process embodiments of the disclosure, nor should variations of user interface methods from those described herein be considered outside the scope of the present disclosure. It is understood that references to displaying or presenting an item (such as, but not limited to, presenting an image on a display device, presenting audio via one or more loudspeakers, and/or vibrating a device) include issuing instructions, commands, and/or signals causing, or reasonably expected to cause, a device or system to display or present the item. In some embodiments, various features described in FIGS. 1-10 are implemented in respective modules, which may also be referred to as, and/or include, logic, components, units, and/or mechanisms. Modules may constitute either software modules (for example, code embodied on a machine-readable medium) or hardware modules.

In some examples, a hardware module may be implemented mechanically, electronically, or with any suitable combination thereof. For example, a hardware module may include dedicated circuitry or logic that is configured to perform certain operations. For example, a hardware module may include a special-purpose processor, such as a field-programmable gate array (FPGA) or an Application Specific Integrated Circuit (ASIC). A hardware module may also include programmable logic or circuitry that is temporarily configured by software to perform certain operations and may include a portion of machine-readable medium data and/or instructions for such configuration. For example, a hardware module may include software encompassed within a programmable processor configured to execute a set of software instructions. It will be appreciated that the decision to implement a hardware module mechanically, in dedicated and permanently configured circuitry, or in temporarily configured circuitry (for example, configured by software) may be driven by cost, time, support, and engineering considerations.

Accordingly, the phrase “hardware module” should be understood to encompass a tangible entity capable of performing certain operations and may be configured or arranged in a certain physical manner, be that an entity that is physically constructed, permanently configured (for example, hardwired), and/or temporarily configured (for example, programmed) to operate in a certain manner or to perform certain operations described herein. As used herein, “hardware-implemented module” refers to a hardware module. Considering examples in which hardware modules are temporarily configured (for example, programmed), each of the hardware modules need not be configured or instantiated at any one instance in time. For example, where a hardware module includes a programmable processor configured by software to become a special-purpose processor, the programmable processor may be configured as respectively different special-purpose processors (for example, including different hardware modules) at different times. Software may accordingly configure a processor or processors, for example, to constitute a particular hardware module at one instance of time and to constitute a different hardware module at a different instance of time. A hardware module implemented using one or more processors may be referred to as being “processor implemented” or “computer implemented.”

Hardware modules can provide information to, and receive information from, other hardware modules. Accordingly, the described hardware modules may be regarded as being communicatively coupled. Where multiple hardware modules exist contemporaneously, communications may be achieved through signal transmission (for example, over appropriate circuits and buses) between or among two or more of the hardware modules. In embodiments in which multiple hardware modules are configured or instantiated at different times, communications between such hardware modules may be achieved, for example, through the storage and retrieval of information in memory devices to which the multiple hardware modules have access. For example, one hardware module may perform an operation and store the output in a memory device, and another hardware module may then access the memory device to retrieve and process the stored output.

In some examples, at least some of the operations of a method may be performed by one or more processors or processor-implemented modules. Moreover, the one or more processors may also operate to support performance of the relevant operations in a “cloud computing” environment or as a “software as a service” (SaaS). For example, at least some of the operations may be performed by, and/or among, multiple computers (as examples of machines including processors), with these operations being accessible via a network (for example, the Internet) and/or via one or more software interfaces (for example, an application program interface (API)). The performance of certain of the operations may be distributed among the processors, not only residing within a single machine, but deployed across several machines. Processors or processor-implemented modules may be in a single geographic location (for example, within a home or office environment, or a server farm), or may be distributed across multiple geographic locations.

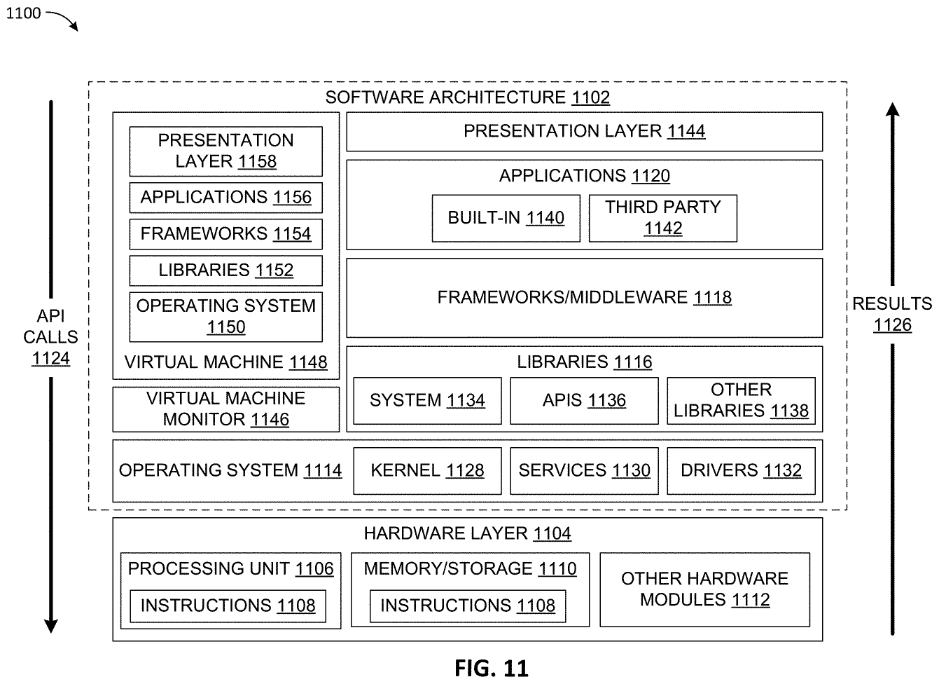

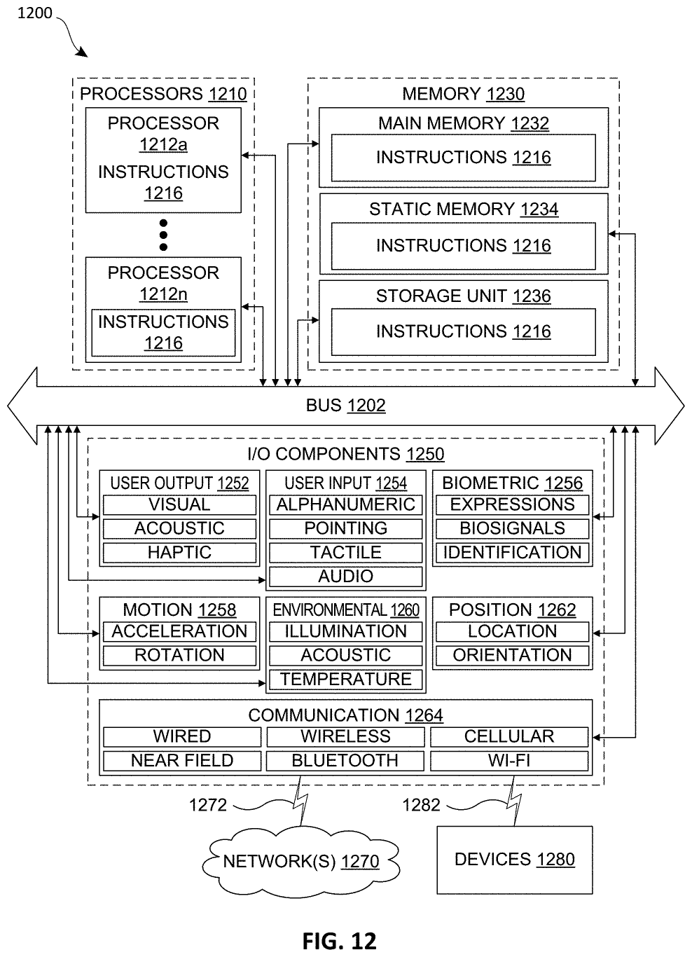

FIG. 11 is a block diagram 1100 illustrating an example software architecture 1102, various portions of which may be used in conjunction with various hardware architectures herein described, which may implement any of the above-described features. FIG. 11 is a non-limiting example of a software architecture, and it will be appreciated that many other architectures may be implemented to facilitate the functionality described herein. The software architecture 1102 may execute on hardware such as a machine 1200 of FIG. 12 that includes, among other things, processors 1210, memory 1230, and input/output (I/O) components 1250. A representative hardware layer 1104 is illustrated and can represent, for example, the machine 1200 of FIG. 12. The representative hardware layer 1104 includes a processing unit 1106 and associated executable instructions 1108. The executable instructions 1108 represent executable instructions of the software architecture 1102, including implementation of the methods, modules and so forth described herein. The hardware layer 1104 also includes a memory/storage 1110, which also includes the executable instructions 1108 and accompanying data. The hardware layer 1104 may also include other hardware modules 1112. Instructions 1108 held by processing unit 1106 may be portions of instructions 1108 held by the memory/storage 1110.

The example software architecture 1102 may be conceptualized as layers, each providing various functionality. For example, the software architecture 1102 may include layers and components such as an operating system (OS) 1114, libraries 1116, frameworks 1118, applications 1120, and a presentation layer 1144. Operationally, the applications 1120 and/or other components within the layers may invoke API calls 1124 to other layers and receive corresponding results 1126. The layers illustrated are representative in nature and other software architectures may include additional or different layers. For example, some mobile or special purpose operating systems may not provide the frameworks/middleware 1118.

The OS 1114 may manage hardware resources and provide common services. The OS 1114 may include, for example, a kernel 1128, services 1130, and drivers 1132. The kernel 1128 may act as an abstraction layer between the hardware layer 1104 and other software layers. For example, the kernel 1128 may be responsible for memory management, processor management (for example, scheduling), component management, networking, security settings, and so on. The services 1130 may provide other common services for the other software layers. The drivers 1132 may be responsible for controlling or interfacing with the underlying hardware layer 1104. For instance, the drivers 1132 may include display drivers, camera drivers, memory/storage drivers, peripheral device drivers (for example, via Universal Serial Bus (USB)), network and/or wireless communication drivers, audio drivers, and so forth depending on the hardware and/or software configuration.

The libraries 1116 may provide a common infrastructure that may be used by the applications 1120 and/or other components and/or layers. The libraries 1116 typically provide functionality for use by other software modules to perform tasks, rather than rather than interacting directly with the OS 1114. The libraries 1116 may include system libraries 1134 (for example, C standard library) that may provide functions such as memory allocation, string manipulation, file operations. In addition, the libraries 1116 may include API libraries 1136 such as media libraries (for example, supporting presentation and manipulation of image, sound, and/or video data formats), graphics libraries (for example, an OpenGL library for rendering 2D and 3D graphics on a display), database libraries (for example, SQLite or other relational database functions), and web libraries (for example, WebKit that may provide web browsing functionality). The libraries 1116 may also include a wide variety of other libraries 1138 to provide many functions for applications 1120 and other software modules.

The frameworks 1118 (also sometimes referred to as middleware) provide a higher-level common infrastructure that may be used by the applications 1120 and/or other software modules. For example, the frameworks 1118 may provide various graphic user interface (GUI) functions, high-level resource management, or high-level location services. The frameworks 1118 may provide a broad spectrum of other APIs for applications 1120 and/or other software modules.

The applications 1120 include built-in applications 1140 and/or third-party applications 1142. Examples of built-in applications 1140 may include, but are not limited to, a contacts application, a browser application, a location application, a media application, a messaging application, and/or a game application. Third-party applications 1142 may include any applications developed by an entity other than the vendor of the particular platform. The applications 1120 may use functions available via OS 1114, libraries 1116, frameworks 1118, and presentation layer 1144 to create user interfaces to interact with users.

Some software architectures use virtual machines, as illustrated by a virtual machine 1148. The virtual machine 1148 provides an execution environment where applications/modules can execute as if they were executing on a hardware machine (such as the machine 1200 of FIG. 12, for example). The virtual machine 1148 may be hosted by a host OS (for example, OS 1114) or hypervisor, and may have a virtual machine monitor 1146 which manages operation of the virtual machine 1148 and interoperation with the host operating system. A software architecture, which may be different from software architecture 1102 outside of the virtual machine, executes within the virtual machine 1148 such as an OS 1150, libraries 1152, frameworks 1154, applications 1156, and/or a presentation layer 1158.

FIG. 12 is a block diagram illustrating components of an example machine 1200 configured to read instructions from a machine-readable medium (for example, a machine-readable storage medium) and perform any of the features described herein. The example machine 1200 is in a form of a computer system, within which instructions 1216 (for example, in the form of software components) for causing the machine 1200 to perform any of the features described herein may be executed. As such, the instructions 1216 may be used to implement modules or components described herein. The instructions 1216 cause unprogrammed and/or unconfigured machine 1200 to operate as a particular machine configured to carry out the described features. The machine 1200 may be configured to operate as a standalone device or may be coupled (for example, networked) to other machines. In a networked deployment, the machine 1200 may operate in the capacity of a server machine or a client machine in a server-client network environment, or as a node in a peer-to-peer or distributed network environment. Machine 1200 may be embodied as, for example, a server computer, a client computer, a personal computer (PC), a tablet computer, a laptop computer, a netbook, a set-top box (STB), a gaming and/or entertainment system, a smart phone, a mobile device, a wearable device (for example, a smart watch), and an Internet of Things (IoT) device. Further, although only a single machine 1200 is illustrated, the term “machine” includes a collection of machines that individually or jointly execute the instructions 1216.

The machine 1200 may include processors 1210, memory 1230, and I/O components 1250, which may be communicatively coupled via, for example, a bus 1202. The bus 1202 may include multiple buses coupling various elements of machine 1200 via various bus technologies and protocols. In an example, the processors 1210 (including, for example, a central processing unit (CPU), a graphics processing unit (GPU), a digital signal processor (DSP), an ASIC, or a suitable combination thereof) may include one or more processors 1212a to 1212n that may execute the instructions 1216 and process data. In some examples, one or more processors 1210 may execute instructions provided or identified by one or more other processors 1210. The term “processor” includes a multi-core processor including cores that may execute instructions contemporaneously. Although FIG. 12 shows multiple processors, the machine 1200 may include a single processor with a single core, a single processor with multiple cores (for example, a multi-core processor), multiple processors each with a single core, multiple processors each with multiple cores, or any combination thereof. In some examples, the machine 1200 may include multiple processors distributed among multiple machines.

The memory/storage 1230 may include a main memory 1232, a static memory 1234, or other memory, and a storage unit 1236, both accessible to the processors 1210 such as via the bus 1202. The storage unit 1236 and memory 1232, 1234 store instructions 1216 embodying any one or more of the functions described herein. The memory/storage 1230 may also store temporary, intermediate, and/or long-term data for processors 1210. The instructions 1216 may also reside, completely or partially, within the memory 1232, 1234, within the storage unit 1236, within at least one of the processors 1210 (for example, within a command buffer or cache memory), within memory at least one of I/O components 1250, or any suitable combination thereof, during execution thereof. Accordingly, the memory 1232, 1234, the storage unit 1236, memory in processors 1210, and memory in I/O components 1250 are examples of machine-readable media.

As used herein, “machine-readable medium” refers to a device able to temporarily or permanently store instructions and data that cause machine 1200 to operate in a specific fashion, and may include, but is not limited to, random-access memory (RAM), read-only memory (ROM), buffer memory, flash memory, optical storage media, magnetic storage media and devices, cache memory, network-accessible or cloud storage, other types of storage and/or any suitable combination thereof. The term “machine-readable medium” applies to a single medium, or combination of multiple media, used to store instructions (for example, instructions 1216) for execution by a machine 1200 such that the instructions, when executed by one or more processors 1210 of the machine 1200, cause the machine 1200 to perform and one or more of the features described herein. Accordingly, a “machine-readable medium” may refer to a single storage device, as well as “cloud-based” storage systems or storage networks that include multiple storage apparatus or devices. The term “machine-readable medium” excludes signals per se.

The I/O components 1250 may include a wide variety of hardware components adapted to receive input, provide output, produce output, transmit information, exchange information, capture measurements, and so on. The specific I/O components 1250 included in a particular machine will depend on the type and/or function of the machine. For example, mobile devices such as mobile phones may include a touch input device, whereas a headless server or IoT device may not include such a touch input device. The particular examples of I/O components illustrated in FIG. 12 are in no way limiting, and other types of components may be included in machine 1200. The grouping of I/O components 1250 are merely for simplifying this discussion, and the grouping is in no way limiting. In various examples, the I/O components 1250 may include user output components 1252 and user input components 1254. User output components 1252 may include, for example, display components for displaying information (for example, a liquid crystal display (LCD) or a projector), acoustic components (for example, speakers), haptic components (for example, a vibratory motor or force-feedback device), and/or other signal generators. User input components 1254 may include, for example, alphanumeric input components (for example, a keyboard or a touch screen), pointing components (for example, a mouse device, a touchpad, or another pointing instrument), and/or tactile input components (for example, a physical button or a touch screen that provides location and/or force of touches or touch gestures) configured for receiving various user inputs, such as user commands and/or selections.

In some examples, the I/O components 1250 may include biometric components 1256, motion components 1258, environmental components 1260, and/or position components 1262, among a wide array of other physical sensor components. The biometric components 1256 may include, for example, components to detect body expressions (for example, facial expressions, vocal expressions, hand or body gestures, or eye tracking), measure biosignals (for example, heart rate or brain waves), and identify a person (for example, via voice-, retina-, fingerprint-, and/or facial-based identification). The motion components 1258 may include, for example, acceleration sensors (for example, an accelerometer) and rotation sensors (for example, a gyroscope). The environmental components 1260 may include, for example, illumination sensors, temperature sensors, humidity sensors, pressure sensors (for example, a barometer), acoustic sensors (for example, a microphone used to detect ambient noise), proximity sensors (for example, infrared sensing of nearby objects), and/or other components that may provide indications, measurements, or signals corresponding to a surrounding physical environment. The position components 1262 may include, for example, location sensors (for example, a Global Position System (GPS) receiver), altitude sensors (for example, an air pressure sensor from which altitude may be derived), and/or orientation sensors (for example, magnetometers).

The I/O components 1250 may include communication components 1264, implementing a wide variety of technologies operable to couple the machine 1200 to network(s) 1270 and/or device(s) 1280 via respective communicative couplings 1272 and 1282. The communication components 1264 may include one or more network interface components or other suitable devices to interface with the network(s) 1270. The communication components 1264 may include, for example, components adapted to provide wired communication, wireless communication, cellular communication, Near Field Communication (NFC), Bluetooth communication, Wi-Fi, and/or communication via other modalities. The device(s) 1280 may include other machines or various peripheral devices (for example, coupled via USB).

In some examples, the communication components 1264 may detect identifiers or include components adapted to detect identifiers. For example, the communication components 1264 may include Radio Frequency Identification (RFID) tag readers, NFC detectors, optical sensors (for example, one- or multi-dimensional bar codes, or other optical codes), and/or acoustic detectors (for example, microphones to identify tagged audio signals). In some examples, location information may be determined based on information from the communication components 1262, such as, but not limited to, geo-location via Internet Protocol (IP) address, location via Wi-Fi, cellular, NFC, Bluetooth, or other wireless station identification and/or signal triangulation.

While various embodiments have been described, the description is intended to be exemplary, rather than limiting, and it is understood that many more embodiments and implementations are possible that are within the scope of the embodiments. Although many possible combinations of features are shown in the accompanying figures and discussed in this detailed description, many other combinations of the disclosed features are possible. Any feature of any embodiment may be used in combination with or substituted for any other feature or element in any other embodiment unless specifically restricted. Therefore, it will be understood that any of the features shown and/or discussed in the present disclosure may be implemented together in any suitable combination. Accordingly, the embodiments are not to be restricted except in light of the attached claims and their equivalents. Also, various modifications and changes may be made within the scope of the attached claims.

While the foregoing has described what are considered to be the best mode and/or other examples, it is understood that various modifications may be made therein and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

Unless otherwise stated, all measurements, values, ratings, positions, magnitudes, sizes, and other specifications that are set forth in this specification, including in the claims that follow, are approximate, not exact. They are intended to have a reasonable range that is consistent with the functions to which they relate and with what is customary in the art to which they pertain.

The scope of protection is limited solely by the claims that now follow. That scope is intended and should be interpreted to be as broad as is consistent with the ordinary meaning of the language that is used in the claims when interpreted in light of this specification and the prosecution history that follows and to encompass all structural and functional equivalents. Notwithstanding, none of the claims are intended to embrace subject matter that fails to satisfy the requirement of Sections 101, 102, or 103 of the Patent Act, nor should they be interpreted in such a way. Any unintended embracement of such subject matter is hereby disclaimed.

Except as stated immediately above, nothing that has been stated or illustrated is intended or should be interpreted to cause a dedication of any component, step, feature, object, benefit, advantage, or equivalent to the public, regardless of whether it is or is not recited in the claims.

It will be understood that the terms and expressions used herein have the ordinary meaning as is accorded to such terms and expressions with respect to their corresponding respective areas of inquiry and study except where specific meanings have otherwise been set forth herein. Relational terms such as first and second and the like may be used solely to distinguish one entity or action from another without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “a” or “an” does not, without further constraints, preclude the existence of additional identical elements in the process, method, article, or apparatus that comprises the element.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various examples for the purpose of streamlining the disclosure. This method of disclosure is not to be interpreted as reflecting an intention that the claims require more features than are expressly recited in each claim. Rather, as the following claims reflect, inventive subject matter lies in less than all features of a single disclosed example. Thus, the following claims are hereby incorporated into the Detailed Description, with each claim standing on its own as a separately claimed subject matter.

What is claimed is:

1. A data processing system comprising:
a processor; and
a machine-readable medium storing executable instructions that, when executed, cause the processor to perform operations comprising:
receiving a set of parameters associated with a first clinical trial, the parameters identifying one or more pharmaceuticals, one or more medical conditions, or both;
identifying first documents associated with one or more second clinical trials based on the parameters associated with the first clinical trial from databases of clinical trials, new drug applications, drug label information, or a combination thereof;
obtaining electronic copies of the first documents;
analyzing the electronic copies using a first set of models configured to identify relevant portions of the electronic copies based on a document type associated with each of the electronic copies;
analyzing the relevant portions of the electronic copies using a natural language processing model to extract information from the relevant portions of the electronic copies;
collating the information extracted from the relevant portions of the electronic copies to produce prediction information related to the first clinical trial; and
analyzing the prediction information to generate one or more reports providing information for assessing aspects of the first clinical trial.

2. The data processing system of claim 1, further comprising:
identifying second documents associated with at least one of drugs determined to be relevant based on the set of parameters, press releases and presentations from organizations determined to be relevant based on the set of parameters, product developments determined to be relevant based on the set of parameters, and business developments determined to be relevant based on the set of parameters; and
obtaining electronic copies of the second documents.

3. The data processing system of claim 1, wherein analyzing the relevant portions of the electronic copies further comprises one or more of:
generating an estimated timeline for the first clinical trial based on timeline information associated with the one or more second clinical trials;
generating a first assessment of endpoints in the one or more second clinical trials, results of studies from earlier phases of drugs associated with the one or more second clinical trials, and evolution of endpoints of the one or more second clinical trials, and a comparison of endpoint outcomes based on mechanisms;
generating a second assessment of comparative performance of drugs based on warnings, contraindications, adverse reactions, administration, and safety concerns;
generating a third assessment of a probability of business success of an organization based on resources, patents, expertise, partnerships, financial status of the organization, and a comparison with similar drug development by that organization or other organizations; and
generating a fourth assessment of a probability of product performance of a drug based on results from past clinical studies of the drug; and
generating a fifth assessment of scenarios of drug performance relating a mechanism of a drug with other mechanisms of other drugs in a disease area and a comparative performance of the first drug and the other drugs.

4. The data processing system of claim 1, wherein collating the information extracted from the relevant portions of the electronic copies further comprises:
clustering the electronic copies into clusters of documents based on trends identified in one or more parameters associated with content of the electronic copies.

5. The data processing system of claim 3, further comprising:
causing to be displayed, on a client device, a dynamic user interface that presents the clusters of documents, the dynamic user interface being configured to present additional details for a respective cluster in response to an input indicating that the respective cluster has been selected.

6. The data processing system of claim 1, wherein the machine-readable medium includes instructions configured to cause the processor to perform operations of:
generating a first model of the first set of models by analyzing a first type of document using a pattern identification algorithm to identify patterns in textual content in the first type of document indicative of the respective relevant portions of a document of the first type.

7. The data processing system of claim 6, wherein the pattern identification algorithm uses Delaunay Triangulation Analogy or Voronoi diagrams Analogy to represent the patterns in the textual content.

8. A method implemented in a data processing system for providing clinical trial recommendations, the method comprising:
receiving a set of parameters associated with a first clinical trial, the parameters identifying one or more pharmaceuticals, one or more medical conditions, or both;
identifying first documents associated with one or more second clinical trials based on the parameters associated with the first clinical trial from databases of clinical trials, new drug applications, drug label information, or a combination thereof;
obtaining electronic copies of the first documents;
analyzing the electronic copies using a first set of models configured to identify relevant portions of the electronic copies based on a document type associated with each of the electronic copies;
analyzing the relevant portions of the electronic copies using a natural language processing model to extract information from the relevant portions of the electronic copies;
collating the information extracted from the relevant portions of the electronic copies to produce prediction information related to the first clinical trial; and
analyzing the prediction information to generate one or more reports providing information for assessing aspects of the first clinical trial.

9. The method of claim 8, further comprising:
identifying second documents associated with at least one of drugs determined to be relevant based on the set of parameters, press releases and presentations from organizations determined to be relevant based on the set of parameters, product developments determined to be relevant based on the set of parameters, and business developments determined to be relevant based on the set of parameters; and
obtaining electronic copies of the second documents.

10. The method of claim 8, wherein analyzing the relevant portions of the electronic copies further comprises one or more of:
generating an estimated timeline for the first clinical trial based on timeline information associated with the one or more second clinical trials;
generating a first assessment of endpoints in the one or more second clinical trials, results of studies from earlier phases of drugs associated with the one or more second clinical trials, and evolution of endpoints of the one or more second clinical trials, and a comparison of endpoint outcomes based on mechanisms;
generating a second assessment of comparative performance of drugs based on warnings, contraindications, adverse reactions, administration, and safety concerns;
generating a third assessment of a probability of business success of an organization based on resources, patents, expertise, partnerships, financial status of the organization, and a comparison with similar drug development by that organization or other organizations; and
generating a fourth assessment of a probability of product performance of a drug based on results from past clinical studies of the drug; and
generating a fifth assessment of scenarios of drug performance relating a mechanism of a drug with other mechanisms of other drugs in a disease area and a comparative performance of the first drug and the other drugs.

11. The method of claim 10, further comprising:
clustering the electronic copies into clusters of documents based on trends identified in one or more parameters associated with content of the electronic copies.

12. The method of claim 10, further comprising:
causing to be display on a client device a dynamic user interface that presents the clusters of documents, the dynamic user interface being configured to present additional details for a respective cluster in response to an input indicating that the respective cluster has been selected.

13. The method of claim 8, further comprising:
generating a first model of the first set of models by analyzing a first type of document using a pattern identification algorithm to identify patterns in textual content in the first type of document indicative of the respective relevant portions of a document of the first type.

14. The method of claim 13, wherein the pattern identification algorithm uses Delaunay Triangles or Voronoi diagrams to represent the patterns in the textual content.

15. A machine-readable medium on which are stored instructions that, when executed, cause a processor of a programmable device to perform operations of:
receiving a set of parameters associated with a first clinical trial, the parameters identifying one or more pharmaceuticals, one or more medical conditions, or both;
identifying first documents associated with one or more second clinical trials based on the parameters associated with the first clinical trial from databases of clinical trials, new drug applications, drug label information, or a combination thereof;
obtaining electronic copies of the first documents;
analyzing the electronic copies using a first set of models configured to identify relevant portions of the electronic copies based on a document type associated with each of the electronic copies;
analyzing the relevant portions of the electronic copies using a natural language processing model to extract information from the relevant portions of the electronic copies;
collating the information extracted from the relevant portions of the electronic copies to produce prediction information related to the first clinical trial; and
analyzing the prediction information to generate one or more reports providing information for assessing aspects of the first clinical trial.

16. The machine-readable medium of claim 15, further comprising instructions configured to cause the processor to perform operations of:
identifying second documents associated with at least one of drugs determined to be relevant based on the set of parameters, press releases and presentations from organizations determined to be relevant based on the set of parameters, product developments determined to be relevant based on the set of parameters, and business developments determined to be relevant based on the set of parameters; and
obtaining electronic copies of the second documents.

17. The machine-readable medium of claim 15, wherein analyzing the relevant portions of the electronic copies further comprises one or more of:
generating an estimated timeline for the first clinical trial based on timeline information associated with the one or more second clinical trials;
generating a first assessment of endpoints in the one or more second clinical trials, results of studies from earlier phases of drugs associated with the one or more second clinical trials, and evolution of endpoints of the one or more second clinical trials, and a comparison of endpoint outcomes based on mechanisms;
generating a second assessment of comparative performance of drugs based on warnings, contraindications, adverse reactions, administration, and safety concerns;
generating a third assessment of a probability of business success of an organization based on resources, patents, expertise, partnerships, financial status of the organization, and a comparison with similar drug development by that organization or other organizations;
generating a fourth assessment of a probability of product performance of a drug based on results from past clinical studies of the drug; and
generating a fifth assessment of scenarios of drug performance relating a mechanism of a drug with other mechanisms of other drugs in a disease area and a comparative performance of the first drug and the other drugs.

18. The machine-readable medium of claim 17, further comprising:
clustering the electronic copies into clusters of documents based on trends identified in one or more parameters associated with content of the electronic copies.

19. The machine-readable medium of claim 15, further comprising instructions configured to cause the processor to perform operations of:
generating a first model of the first set of models by analyzing a first type of document using a pattern identification algorithm to identify patterns in textual content in the first type of document indicative of the respective relevant portions of a document of the first type.

20. The machine-readable medium of claim 19, wherein the pattern identification algorithm uses Delaunay Triangulation or Voronoi diagrams to represent the patterns in the textual content.
